(19) United States
(12) Patent Application Publication
(10) Pub. No.: US 2010/0261775 A1
(43) Pub. Date: Oct. 14, 2010

(54) POLYMORPHIC FORM OF DULOXETINE HYDROCHLORIDE

(76) Inventors: Sujoy Biswas, West Bengal (IN); Keya Karanjai, Uttar Pradesh (IN)

Correspondence Address: Ranbaxy Inc., Intellectual Property Department, 600 College Road East, PRINCETON, NJ, 08540, US

(21) Appl. No.: 12/305,779

(22) PCT Filed: Jul. 3, 2007
(86) PCT No.: PCT/IB2007/052603

§ 371 Date: May 10, 2010

(30) Foreign Application Priority Data
Jul. 3, 2006 (IN) 1554/DEL/2006

(51) Int. Cl. A61K 31/381; C07D 333/20; A61P 25/24

(52) U.S. Cl. 514/438; 549/75

(57) ABSTRACT

The present invention relates to Form I of duloxetine hydrochloride and its preparation.

### FIELD OF THE INVENTION

The present invention relates to a polymorphic form of duloxetine hydrochloride and processes for its preparation. The polymorphic form of the current invention is designated Form I. The present invention also relates to a process for preparing duloxetine hydrochloride from duloxetine maleate.

### BACKGROUND OF THE INVENTION

Duloxetine hydrochloride is a selective serotonin and norepinephrine reuptake inhibitor (SSNRI) for oral administration. It is chemically (+)-(S)-N-methyl-γ-(1-naphthyloxy)-2-thiophenepropylamine hydrochloride as represented by Formula I:

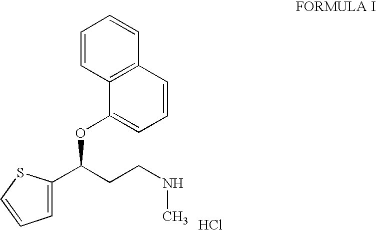

U.S. Pat. No. 5,023,269 provides a process for the preparation of laconic duloxetine oxalate and it discloses maleate and oxalate salts of S-(+)-duloxetine. However, the '269 patent does not provide any method to separate specific enantiomers of duloxetine. U.S. Pat. No 5,491,243 provides a similar process for preparing for duloxetine, wherein the final compound of duloxetine is isolated as a hydrochloride salt using ethyl acetate as a solvent and seeding. The '243 patent says that the desired product is prepared in yields in the range of 95% with very little racemization and that previous procedures gave a product of inferior purity.

PCT application WO 05/019199 provides processes for preparing amorphous duloxetine hydrochloride by vacuum drying methods. WO 05/108386 provides processes for preparing Forms A, B and C of free base of duloxetine.

Various processes for the preparation of duloxetine and its intermediates are also provided in EP 0,457,559 A3, U.S. Pat. No. 5,362,886, WO 03/062219, WO 03/070720, EP 1,506,965, WO 04/005307, US 2004/0181058, WO 04/056795, WO 04/065376, WO 04/055194, WO 03/018572, JP 2003-192681 A2, US 2003/225153, US 2005/107621, WO 04/005220, WO 04/005239, WO 04/011452, WO 04/013123, WO 04/016603, DE 10237272 A1, WO 04/020389, WO 04/024708, WO 04/031168, EP 1411045 A1, DE 10248479 A1, DE 10248480 A1, WO 04/090094, WO 04/103990, WO 05/021527, WO 05/033094, WO 05/073215, WO 05/080370, US 2003/225274, US 2003/225153, US 2004/023348, US 2004/023344, U.S. Pat. No. 6,924,386, DE 10237272 A1, US 2004/181058.

### BRIEF DESCRIPTION OF THE FIGURES

FIG. 1 is an X-ray powder diffractogram (XRPD) pattern of Form I of duloxetine hydrochloride.

FIG. 2 is a Fourier-Transform Infra-red (FTIR) spectrum of Form I of duloxetine hydrochloride.

### SUMMARY OF THE INVENTION

The present invention provides duloxetine hydrochloride Form I, which is suitable for preparing pharmaceutical dosage forms. The present invention further provides a process for preparing Form I of duloxetine hydrochloride. The present inventors have also developed a simple and efficient process for preparing duloketine hydrochloride from duloxetine maleate.

### DETAILED DESCRIPTION OF THE INVENTION

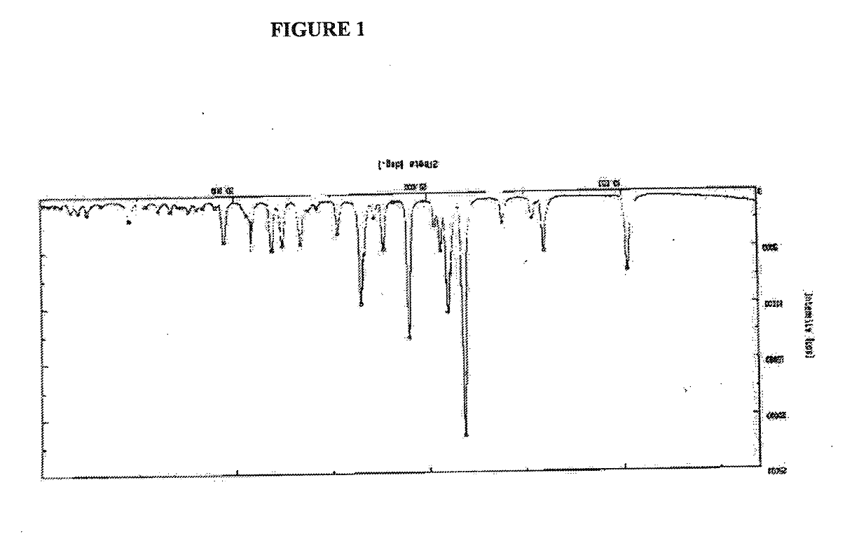

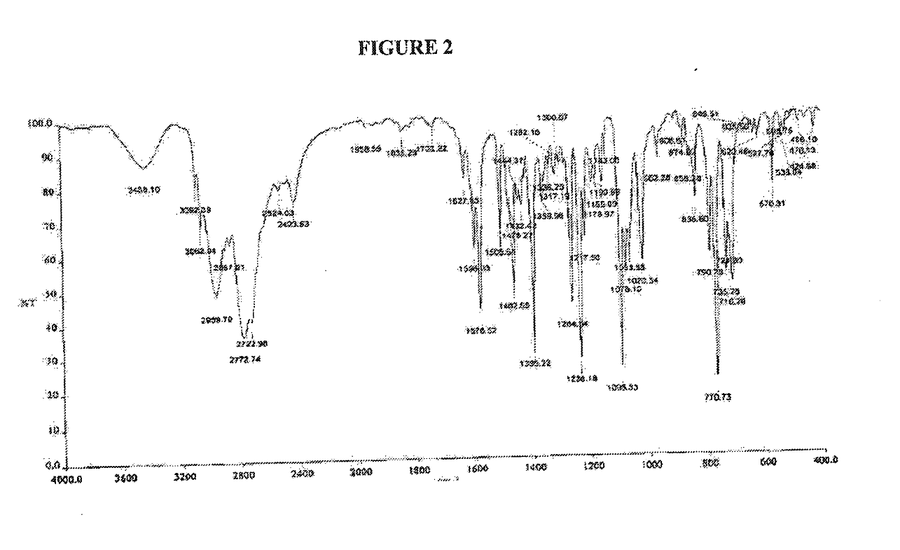

In one aspect, Form I of duloxetine hydrochloride is provided, having, for example, XRPD pattern substantially as provided, for example, in FIG. 1. The XRPD pattern of Form I of duloxetine hydrochloride can be characterized by peaks at 2θ values 9.74, 14.02, 18.20, 18.86, 19.02, 21.00, 22.28, 23.28, 23.48 and 24.64±0.2. It is further characterized by additional peaks at 2θ values at 14.62, 16.14, 19.36, 19.64, 20.16, 21.46, 21.72, 22.74, 25.72, 26.16, 26.58, 27.52, 28.08, 29.1, 29.36 and 30.5+0.2. A representative FTIR spectrum of Form I of duloxetine hydrochloride is provided in FIG. 2.

In another aspect, a process for the direct preparation of duloxetine hydrochloride from duloxetine maleate without the need for seeding is provided, wherein the process comprises,

a) treating duloxetine maleate with a base to obtain free base of duloxetine,

b) contacting the free base of duloxetine with hydrochloric acid, and

c) isolating duloxetine hydrochloride from the reaction mixture.

The duloxetine maleate can be prepared, for example, according to the method provided in Tetrahedron Letters 1990, 31(49), 7101-7104. The maleate salt of duloxetine is treated with a base in the presence of water or water miscible organic solvent, or a mixture thereof. An alkali metal hydroxide is preferably used as the base. The liberated free base of duloxetine is extracted with a water immiscible organic solvent. The water immiscible organic solvent is preferably an aromatic hydrocarbon. According to the processes described herein, the free base of duloxetine is not required to be isolated from the organic solvent and it is contacted with hydrochloric acid after partially concentrating the solution. Hydrochloric acid may be used as a gas or as a solution in water or organic solvents. The duloxetine hydrochloride may be isolated from the reaction mixture by solvent precipitation, concentration, distillation and other such conventional techniques.

In yet another aspect, a process for the preparation of Form I of duloxetine hydrochloride is provided, wherein the process comprises,

a) dissolving duloxetine hydrochloride in a solvent,

b) treating the solution obtained in step (a) with an anti-solvent, and

c) isolating Form I of duloxetine hydrochloride from the reaction mixture.

Duloxetine hydrochloride—in any previously known crystalline or amorphous form—prepared by methods known in the art can be used as the starting material. Duloxetine hydrochloride can be dissolved in an organic solvent or a mixture of an organic solvent and water. The organic solvent can be, for example, a C₁₋₃ alkanol, acetonitrile, acetone, dioxane, dimethyl formamide or tetrahydrofuran. The organic solvent can be, for example, absolute ethanol. The duloxetine hydrochloride can be dissolved by heating the mixture from about 40° C. to about 80° C. An anti-solvent may be added to the solution so obtained. The anti-solvent can be, for example, an aliphatic ether, aliphatic hydrocarbon, aromatic hydrocarbon or aliphatic ester. The reaction mixture can be initially heated to a temperature of about 50° C. and then cooled to 35° C. or below to obtain Form I of duloxetine hydrochloride.

In still another aspect, a pharmaceutical composition comprising Form I of duloxetine hydrochloride is provided which optionally contains one or more excipients.

In yet a further aspect, a method for inhibiting serotonin uptake in mammals is provided which comprises administering a pharmaceutically effective amount of Form I of duloxetine hydrochloride to a mammal in need of treatment with a serotonin uptake inhibitor.

Powder XRD of the samples were determined by using X-Ray diffractometer, Rigaku Corporation, RU-H3R, Goniometer CN2155A3, X-Ray tube with Cu target anode, Power: 40 KV, 100 mA, Scanning speed: 2 deg/min step: 0.02 deg, Wave length: 1.5406 Å

FTIR spectra of the samples were recorded on a Perkin-Elmer 16 PC instrument, as potassium bromide pellets.

While the present invention has been described in terms of its specific embodiments, certain modifications and equivalents will be apparent to those skilled in the art and are intended to be included within the scope of the present invention.

### EXAMPLES

### Example 1

### Preparation of Duloxetine Hydrochloride

a) Preparation of duloxetine:

A suspension of duloxetine maleate (20 g) in water was basified to about pH 12 using 30% aqueous sodium hydroxide solution at about 25° C. The reaction mixture was extracted with toluene (2×200 mL). The toluene layer was washed with water till the pH was between 7 and 8 and then concentrated under reduced pressure to obtain the title compound as an oily mass which was used directly in the following step.

b) Preparation of Duloxetine Hydrochloride

The oily mass obtained in step (a) was dissolved in ethyl acetate (90 mL). The pH of the solution was adjusted to between 1.5 and 2.0 using a solution of hydrochloric acid in ethyl acetate [Assay ˜8% (w/w)] at 5-10° C. to attain the pH of 1.5 to 2.0. The reaction mixture was stirred at 5°-10° C. for 2 h. The resultant solid was filtered, washed with ethyl acetate (2×20 mL) and dried under vacuum at 45°-50° C. for 8-10 h to obtain the title compound as an off-white solid.

Yield: 14 g

### Example 2

### Preparation of Form 1 of Duloxetine Hydrochloride

A mixture of duloxetine hydrochloride obtained as prepared in Example 1 (10 g) in absolute ethanol (30 mL) was stirred at 65°-70° C. for 15 minutes to obtain a clear solution. Activated charcoal (1.0 g) was added to the solution so obtained and stirred at 65°-70° C. for further 30 minutes. The charcoal was filtered and washed with absolute ethanol (3×15 mL) at about 25° C. Diisopropyl ether was added (35 mL) to the combined filtrate and washed at 40°-45° C. The reaction mixture was reheated to 65°-68° C. for 15 minutes and cooled to 25°-30° C. to obtain a white solid as a precipitate. The mixture was stirred at 25°-30° C. for 2 h and further at 5°-10° C. for 2 h. The solid was filtered, washed with a mixture of absolute ethanol (7.5 mL) and diisopropyl ether (7.5 mL) at about 25° C. and dried under vacuum at 45°-50° C. for 8 to 1.0 h to obtain the title compound having XRPD and FTIR patterns as depicted in FIGS. 1 and 2 respectively.

Yield: 7.5 g

Enantiomeric purity: 99.99%.

We claim:

1. Form I of duloxetine hydrochloride having an XRPD pattern substantially as depicted in FIG. 1.

2. Form I of duloxetine hydrochloride having peaks at substantially the following 2θ values in the XRPD plot: 9.74, 14.02, 18.20, 18.86, 19.02, 21.00, 22.28, 23.28, 23.48, and 24.64±0.2.

3. Form I of duloxetine hydrochloride having substantially an FTIR spectrum as depicted in FIG. 2.

4. A process for the preparation of Form I of duloxetine hydrochloride, comprising:
a) dissolving duloxetine hydrochloride in a solvent;
b) treating the solution obtained in step (a) with an anti-solvent; and
c) isolating Form I of doloxetine hydrochloride from the reaction mixture.

5. A process according to claim 4, wherein the solvent is at least one of a C₁₋₃ alkanol, acetonitrile, acetone, dioxane, dimethyl formamide or tetrahydrofuran.

6. A process according to claim 5, wherein the solvent is absolute ethanol.

7. A process according to claim 4, wherein the anti-solvent is at least one of an aliphatic ether, aliphatic hydrocarbon, aromatic hydrocarbon or aliphatic ester.

8. A pharmaceutical composition comprising Form I of duloxetine hydrochloride.

9. A method for inhibiting serotonin uptake in mammals which comprises administering a pharmaceutically effective amount of Form I of duloxetine hydrochloride to a mammal in need of treatment with a serotonin uptake inhibitor.

10. A process for the preparation of duloxetine hydrochloride, comprising:
a) treating duloxetine maleate with a base to obtain free base of duloxetine;
b) contacting the free base of duloxetine with hydrochloric acid; and
c) isolating duloxetine hydrochloride from the reaction mixture, without using any seed.

11. A process according to claim 5, wherein the base is an alkali metal hydroxide.

12. A. process according to claim 5, wherein step a) is carried out in the presence of water or water miscible organic solvent, or a mixture thereof.

13. A process according to claim 7, wherein step a) further comprises extracting the free base of duloxetine with a water immiscible organic solvent.
